(19) United States
(12) Patent Application Publication
(10) Pub. No.: US 2010/0030327 A1
(43) Pub. Date: Feb. 4, 2010

(54) DEVICE FOR THE IMPLANTATION OF A THERAPEUTIC OR DIAGNOSTIC APPARATUS IN OR ON A MAMMALIAN INTERNAL ORGAN

(76) Inventor: Didier Chatel, Mettray (FR)

Correspondence Address: BROWDY AND NEIMARK, P.L.L.C.;624 NINTH STREET, NW, SUITE 300, WASHINGTON, DC, 20001-5303, US

(21) Appl. No.: 12/445,372

(22) PCT Filed: Oct. 11, 2007
(86) PCT No.: PCT/IB07/04167

§ 371 Date: Apr. 13, 2009

Related U.S. Application Data
(60) Provisional application No. 60/857,789, filed on Nov. 9, 2006.

(30) Foreign Application Priority Data
Oct. 13, 2006 (FR) 0654267

(51) Int. Cl. A61F 2/24; A61B 17/00; A61M 5/158

(52) U.S. Cl. 623/2.11; 600/37; 606/129; 604/176

(57) ABSTRACT

Device (1) for the implantation of an apparatus (40) on or in a mammalian internal organ, comprising: —a tube (10) for passing the apparatus through, one end (11) of which is intended to be applied to a site chosen for the implantation of the apparatus, and the other end of which is intended to emerge outside the body of the mammal, —fixing means (20) suitable for fixing the device on the organ and for applying the end of the tube to the chosen site, said means being controlled from outside the body, —rigidifying means (30) suitable for rigidifying the device, said means being controlled from outside the body, so as to fix the position of the tube relative to the fixing means and to the organ, once the device has been fixed on the organ and the end of the tube has been applied to the chosen site by the fixing means.

The present invention relates to a device for the implantation of an apparatus in or on a mammalian internal organ.

More particularly, the present invention relates to a device for the implantation of a therapeutic or diagnostic apparatus.

Medical progress, and surgical progress in particular, is aimed at developing procedures (diagnostic and/or therapeutic) that are relatively non-invasive and therefore not very aggressive, so as to satisfy new needs in public health: new needs, in particular, due to the constant aging of the population.

In this context, a certain number of surgical procedures no longer require opening of the thorax, and are now carried out on a closed thorax, the elements for performing the procedure being introduced through orifices made in the thoracic wall for this purpose. The drawbacks associated with a thoracotomy (pain, scars, prolonged hospitalization) are thus avoided. Rational specific tools are increasingly required for the implementation of these new techniques.

Heart failure, for example, is marked, in a certain number of cases, by a loss of synchronism between the contractions of the right ventricle and those of the left ventricle. Cardiac resynchronization is a therapeutic solution aimed at optimizing the mechanical effectiveness of the heart, and consists in implanting electrostimulation probes in the heart chambers or at the surface of the heart. If implantation of one of these probes via the veins of the organism (endovascular approach) fails, said probe must be implanted surgically by opening the thorax (thoracotomy) and placing the probe on the heart (epicardial implantation) via surgical sutures or by means of specific implantation tools.

In order to avoid the drawbacks of performing a thoracotomy, certain surgical implantations of epicardial electrostimulation probes can be carried out with a closed thorax by means of specific tools under video control (with endoscopy cameras); this is then referred to as video-assisted thoracoscopic implantation. All the elements are introduced into the thorax through orifices made in the thoracic wall with trocars and mandrins. The current design of specific tools for video-assisted thoracoscopic implantation remains imperfect, limiting their functionality and consequently their procedural effectiveness, all the more so since certain optimal target sites for epicardial implantation can be difficult to access with these current tools. Moreover, the target site for epicardial implantation, i.e. the site where the electrostimulation will make it possible to obtain the best clinical benefit of the resynchronization, must sometimes be selected by the surgeon through repetitive electrostimulation trials on various sites judged to be potentially effective. Now, no current tool makes it possible to carry out such trials without risking damage to the surface of the heart and therefore possible complications in the procedure.

The present invention aims to overcome these drawbacks. To this effect, it proposes a device for the implantation of an apparatus on or in a mammalian internal organ, comprising:
a tube for passing the apparatus through, one end of which is intended to be applied to a site chosen for the implantation of the apparatus, and the other end of which is intended to emerge outside the body of the mammal,
fixing means suitable for fixing the device on the organ and for applying the end of the tube to the chosen site, said means being controlled from outside the body,
rigidifying means suitable for rigidifying the device, said means being controlled from outside the body, so as to fix the position of the tube relative to the fixing means and to the organ, once the device has been fixed on the organ and the end of the tube has been applied to the chosen site by the fixing means.

Thus, the invention proposes an instrument that is sufficiently flexible to be introduced and manipulated in the body, and that becomes sufficiently rigid and fixed on an organ to allow the precise implantation, at a selected site of this organ, of a diagnostic and/or therapeutic apparatus. In particular, by means of this device, it is possible to implant a cardiac electrostimulation probe at the surface of the heart (epicardium) or in the wall of the heart (myocardium).

Advantageously, the device according to the invention is capable of having autonomous stability, once it is fixed on the organ. More particularly, the device adheres to the organ and maintains the tube in position with respect to the organ by its own means. It is thus not necessary to hold the device in place by other means, the device having autonomous adhesion and stability with respect to the organ. The device may thus be completely let free once it is positioned on the organ, the device being autonomous to hold its position and its orientation with respect to the organ.

The device according to the invention makes it possible to dispense with maintaining means other than the fixing and rigidifying means with which it is provided. Advantageously, this enables the constraint on the organ during the procedure to be reduced.

Advantageously, in the context of a procedure on an organ that is constantly moving, such as the heart or the lungs, the invention makes it possible to be clear of the movements of the organ (heartbeats, for example) and to stabilize the apparatus before implanting it.

According to preferred arrangements of the invention, in particular for reasons of convenience, effectiveness and reliability:
the fixing means are a suction cup placed around said end of the tube intended to be applied to the organ, without communicating with the interior of the tube, and provided with a suction line for generating a vacuum under the suction cup so as to be able to cause the suction cup to adhere to the organ and thus to be able to fix the end of the tube at the chosen site on the organ for the implantation of the apparatus;
the rigidifying means are a circular sack placed around a portion of the tube in the region of the end of the tube intended to be applied to the organ, the sack being closed at its first end around the tube, and, at its other end, closed over an annular portion of the fixing means, the sack being provided with a suction line for generating a vacuum inside the sack, and being designed so that it is flexible when the pressure inside it is the same as the surrounding pressure, and so that, when the vacuum is generated inside it, it tightens around the tube so as to fix the position of the tube that passes through it, relative to the fixing means, and to contribute to rigidifying the device;
the sack is filled with a plurality of solids, free in the sack, so that, when the vacuum is generated in the sack, the sack with the solids tightens around the tube so as to contribute to fixing the position of the tube that passes through the sack, relative to the fixing means, and to contribute to the rigidification of the device;
the sack has bumps on its inner surface, suitable for contributing to fixing the position of the tube relative to the fixing means and to the organ when the vacuum is generated in the sack;
the sack has bumps on its outer surface, suitable for contributing to fixing the position of the tube relative to the fixing means and to the organ when the vacuum is generated in the sack;
the end of the tube intended to be applied to the internal organ is provided with a ring made of flexible material aimed at making this end non-traumatic;
the apparatus to be implanted is a hollow needle designed to inject a product into the organ;
the device is intended for a human organ;
the device is intended for a heart;
the apparatus to be implanted is a cardiac stimulation probe;
the apparatus to be implanted is a heart valve prosthesis;
the end of the tube for passing the probe through, and the suction cup placed around the end of the tube, are intended to be applied to the epicardium.

According to a preferred aspect, in a device according to the invention:
the apparatus is a trocar guide carrying a heart valve prosthesis set on this trocar guide and intended to be implanted in the aortic valve position by expansion,
the fixing means are a suction cup, placed around the end of the tube, and suitable for being applied to the apex of the heart,
the position of the tube is intended to be fixed, relative to the fixing means and to the organ, in an orientation such that the trocar supporting the heart valve prosthesis is stabilized along an axis compatible with an anatomically and physiologically effective implantation of the heart valve prosthesis.

According to a preferred aspect, the device according to the invention is intended to be used in cardiac therapy.

According to a preferred aspect, the device according to the invention is adapted to be used in a procedure consisting of:
introducing the device into the body to treat via an introduction orifice;
applying the device to a site of the surface of the organ to treat;
generating the vacuum under the suction cup via the vacuum line of the suction cup;
choosing an angle of inclination of the tube with respect to the surface of the organ to treat;
generating the vacuum in the sack via the vacuum line of the sack;
implanting the therapeutic or diagnostic apparatus at the surface of the organ to treat after having introduced said apparatus by the tube.

According to a preferred aspect of the invention, the device is adapted to be used in a procedure consisting of:
introducing the device into the body to treat via the introduction orifice;
applying the device to a site of the surface of the organ to treat;
generating the vacuum under the suction cup via the vacuum line of the suction cup;
choosing an angle of inclination of the tube with respect to the surface of the organ to treat;
generating the vacuum in the sack via the vacuum line of the sack;
implanting the therapeutic or diagnostic apparatus at the surface of the organ to treat after having introduced said apparatus by the tube;
conducting tests on the parameters of the apparatus implanted at said site of the surface of the organ to treat;
depending on the results of said tests:
either, if said results are satisfactory, implanting the apparatus definitively, releasing the vacuum in the sack and under the suction cup, and withdrawing the implantation device, leaving the apparatus definitively implanted on or in the organ.
if not, withdrawing the apparatus, releasing the vacuum in the sack and under the suction cup, repositioning the latter and again generating the vacuum under the suction cup so as to fix it onto another site of the surface of the organ to treat, positioning the tube with respect to the surface of the organ to treat, generating the vacuum in the sack via the vacuum line of the sack, implanting the apparatus at the surface of the organ, conducting tests on the parameters of the implanted apparatus; and repeating these operations until satisfactory parameters of the apparatus are obtained.

Advantageously, the fact of having the possibility of controlling the two vacuum lines separately, and thus of being able to proceed in two steps, respectively to position the suction cup via the vacuum line of the suction cup, then to rigidify the sack via the vacuum line of the sack, makes it possible to reduce the risk of loss of vacuum under the suction cup at the time of the manipulations necessary for the choice of the inclination of the tube with respect to the surface of the organ. More particularly, while the sack is not rigidified, the tube may be manipulated freely without risking detaching the suction cup from the surface of the organ.

It is only after the choice of this positioning that the position of the tube is fixed with respect to the surface of the organ, by rigidifying the sack.

Other characteristics and advantages of the present invention will emerge more clearly on reading the description of an embodiment of a device according to the invention that follows, given by way of illustration that is no way limiting, with reference to the attached schematic drawings, in which:

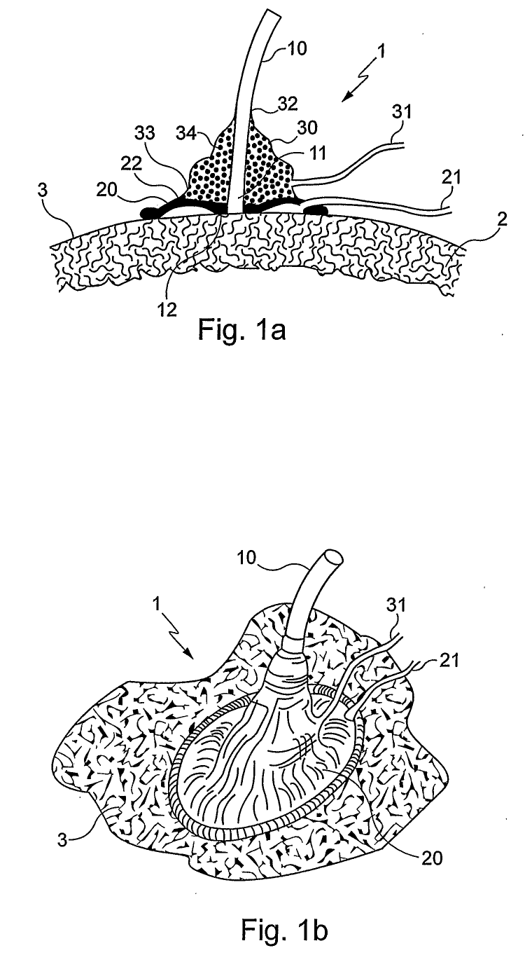

FIGS. 1a and 1b are respectively sectional and perspective views of the same device according to the invention in the position of application on a heart,

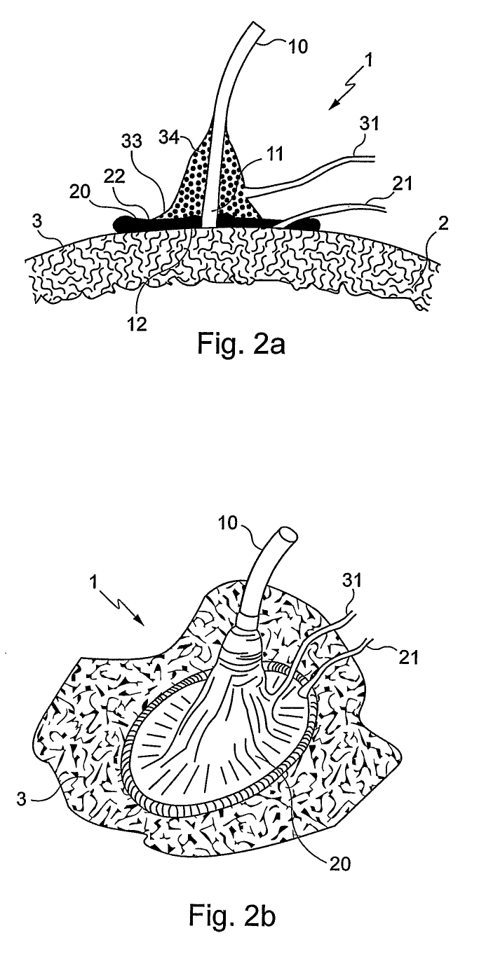

FIGS. 2a and 2b are respectively sectional and perspective views of the same device according to the invention in the position fixed on the heart,

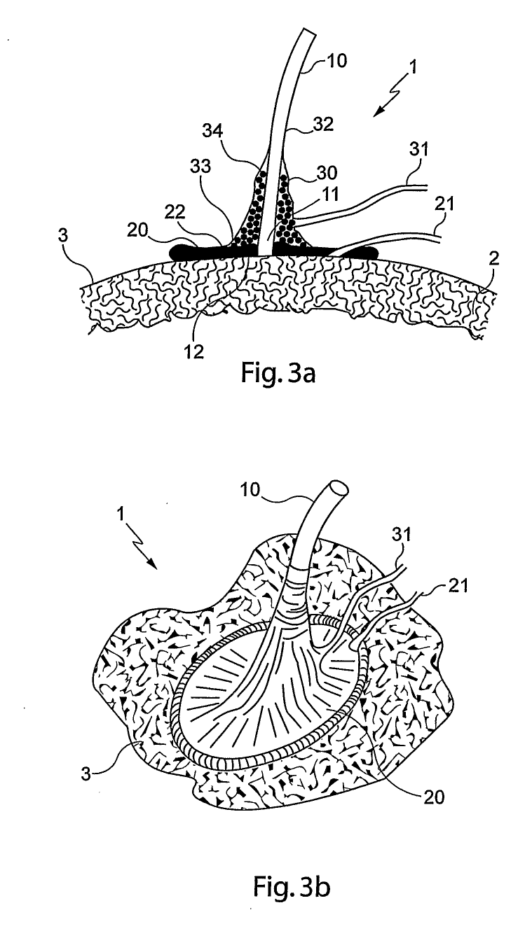

FIGS. 3a and 3b are respectively sectional and perspective views of the same device according to the invention in the position fixed and rigidified on the heart,

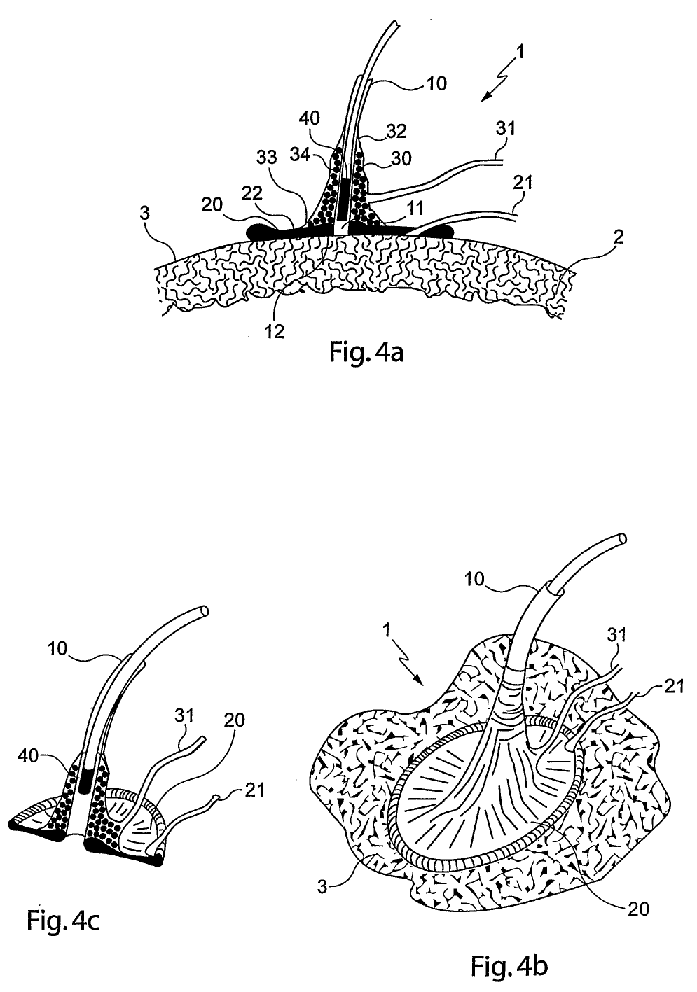

FIGS. 4a and 4b are respectively sectional and perspective views of a device according to the invention in the position of application on the heart,

FIG. 4c is a sectional view of the device represented in FIG. 4b,

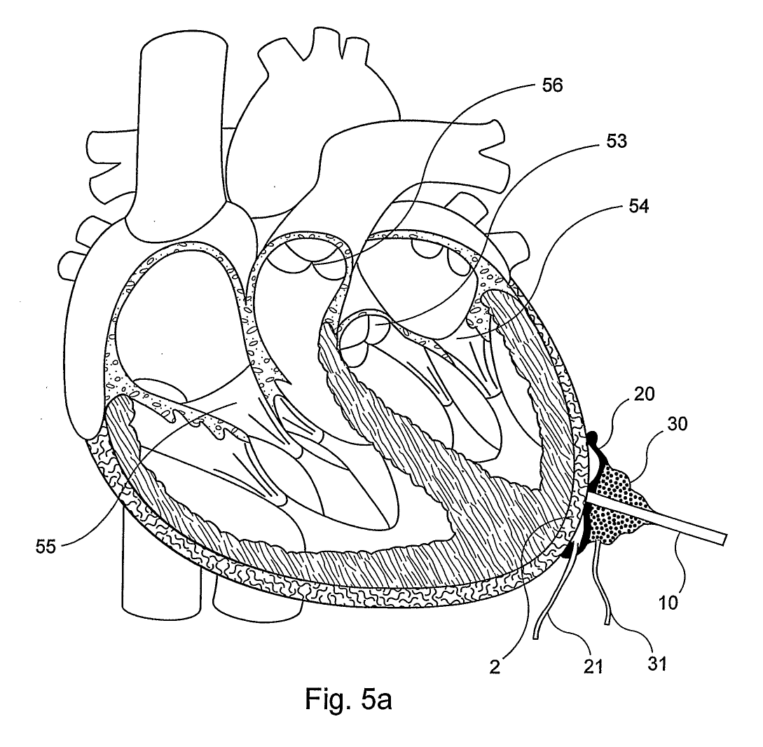

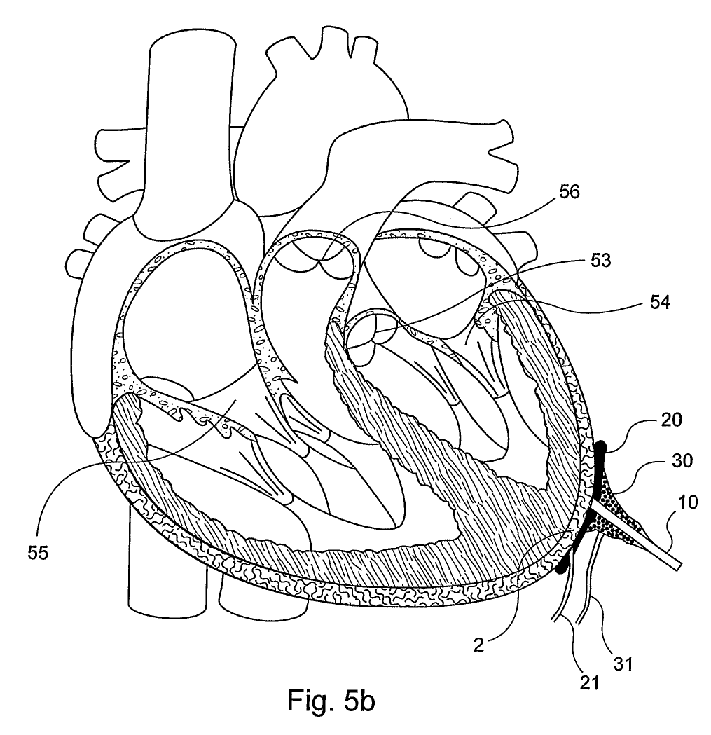

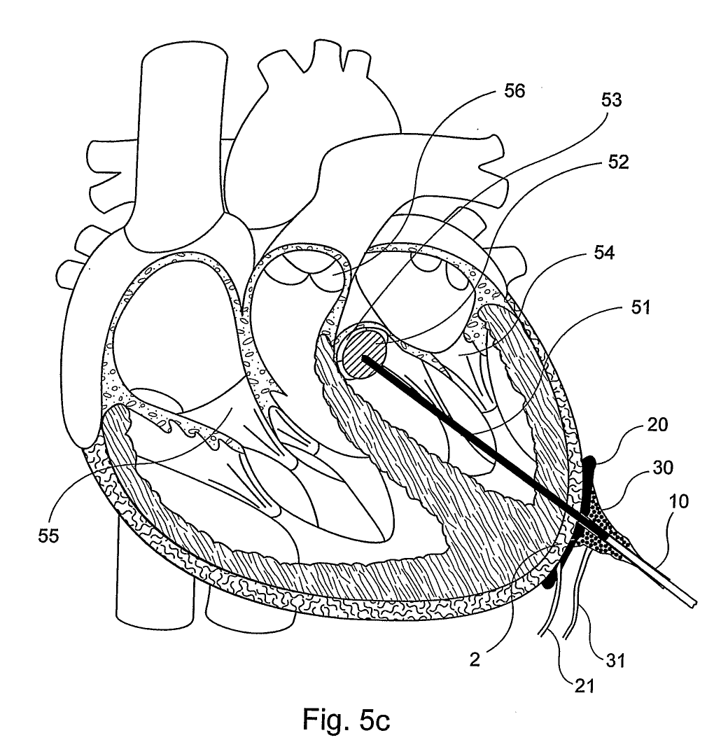

FIGS. 5a to 5c are very schematic sectional views of a device according to the invention suitable for the implantation of a heart valve prosthesis in the aortic valve position in a human heart, the device being respectively in the approach position, in the fixed position and in the working rigidified position on the heart.

As is visible in particular in FIGS. 1a and 1b, a device 1, according to the invention, intended to be affixed on the epicardium 3 of a heart 2, has been represented. The device 1 comprises a tube 10 for passing through an apparatus to be implanted, a suction cup 20 placed around one end 11 of the tube 10, intended to applied to the epicardium, and a sack 30 placed around the same tube.

The suction cup 20 is provided with a suction line 21 to generate a vacuum therein so as to fix the suction cup onto the epicardium 3.

The tube 10 for passing the apparatus through is provided, at its end 11 intended to be applied to the epicardium, with a flexible ring 12 for avoiding any damage to the epicardium and improving the airtightness for the creation of the vacuum under the suction cup 20.

The sack 30 has a circular cross section overall. It is placed around the tube 10 so as to close at a first end 32 and it is closed over an annular portion 22 of the suction cup 20 at its other end 33.

The sack 30 is provided with a suction line 31 so as to generate the vacuum therein. The sack 30 is also filled with a plurality of solids 34, such that, when the vacuum is generated therein, the sack tightens around the tube 10 so as to hem in the solids and fix the position of the tube 10 relative to the suction cup 20, and to rigidify the device 1. When the vacuum is generated in the sack 30, the solids 34 tightened by the sack 30 against the tube 10 contribute to the rigidity of the device 1.

In practice, during the first phases of a surgical procedure, the suction lines 21 and 31 are not solicited, and the device 1 remains flexible. It is then introduced into the patient's body via an introduction orifice. The device 1 is then applied to the epicardium 3 on a site where the cardiac therapy apparatus will be implanted.

The vacuum is then generated under the suction cup 20 via the suction line 21, and the device 1 is flattened against and fixed onto the epicardium, as visible in FIGS. 2a and 2b. The flexible ring 12 placed at the end 11 of the tube 10 contributes to the airtightness so as to maintain the vacuum under the suction cup 20 and makes it possible to avoid damaging the epicardium at this site, by making this end 11 of the tube non-traumatic.

At this stage, the tube 10 still has a certain freedom of movement relative to the suction cup 20 and the epicardium 3. This allows the surgeon to choose an angle of inclination of the tube 10 relative to the epicardium 3. Once this angle is chosen, the vacuum is generated in the sack 30 via the suction line 31. Thus, the sack 30 tightens the solids 34 with respect to one another and against the tube 10 so as to rigidify the device 1 and to fix the position of the tube 10 relative to the suction cup 20, itself fixed relative to the epicardium 3. In this position, which is more particularly visible in FIGS. 3a and 3b, the device 1 is rigidified and is ready to receive the cardiac therapy or diagnosis apparatus intended to be implanted.

The choice of the angle of implantation is particularly important when a probe is placed in the epicardium. This is because, if the probe is implanted in the myocardium perpendicular to the surface of the heart, there is a risk of piercing the myocardium although the contact surface between the probe and said myocardium remains small. On the other hand, if a more acute angle of attack is chosen (an angle tangential to the surface of the heart), it is possible to increase the contact surface between the probe and the myocardium without, however, risking piercing the latter. Advantageously, the device according to the invention makes it possible to position the tube so as to implant the probe with an optimized angle.

As is visible more particularly in FIGS. 4a to 4c, a cardiac therapy apparatus, in this case a stimulation probe 40, is introduced via the tube 10 until it reaches the end 11 for implantation in the myocardium through the epicardium 3.

In the present preferred embodiment, and in particular in the case of a surgical procedure aimed at cardiac resynchronization, the positioning of the stimulation probe on the epicardium cannot be determined in advance, as previously specified. The surgeon must therefore carry out, during the procedure, provisional implantations of the stimulation probe 40 so as to test the effects thereof on the heart. The device 1 according to the invention as visible in FIGS. 4a to 4c creates favorable conditions for the provisional implantation of a stimulation probe. The surgeon then carries out trials regarding the electrical parameters and, depending on the results, implants the probe definitively or withdraws the stimulation probe. In these two cases, respectively, the surgeon then carries out the following procedures:

a) Implant the probe definitively, release the vacuum in the sack and under the suction cup, and withdraw the implantation device, leaving the probe definitively implanted, if the electrical parameters are optimal for the desired stimulation. In this case, the implantation device slides along the stimulation probe so as to be removed from the patient's thorax.

b) Withdraw the stimulation probe, release the vacuum in the sack and under the suction cup, reposition the latter and again generate the vacuum so as to fix it onto the epicardium, generate the vacuum in the sack once the positioning of the tube relative to the epicardium has been chosen, and again introduce therein the stimulation probe for a further trial. The procedures are repeated until the surgeon finds a satisfactory placement for the implantation.

It will be noted that the device according to the invention makes it possible to make the surgical procedure more brief, less invasive and less of an impairment to the health, and, consequently, allows a reduction in hospitalization time.

Moreover, it will be noted that all surgical approaches are possible with a device according to the invention, and in particular a closed-thorax approach under the control of a video camera (video-assisted thoracoscopic approach).

Finally, it will be noted that the device according to the invention allows a ready and reliable repositioning of the probe, without tissue damage, during provisional implantation for obtaining better electrical parameters.

By virtue of these characteristics, the device according to the invention makes it possible to obtain better clinical results at lower human and economic costs.

In a variant that is not illustrated, bumps are provided on the outer surface of the sack, these bumps contributing to fixing the position of the portion of the tube that passes through the sack, relative to the suction cup, and thus contributing to the rigidification and to the stability of the device when the vacuum is generated therein.

According to another aspect of this embodiment of the invention, illustrated in FIGS. 5a to 5c, the device is suitable for implanting a heart valve prosthesis 52 in the aortic valve position 53.

The apparatus is, in this case, a trocar guide 51 carrying a heart valve prosthesis 52 (represented very schematically in FIG. 5c) set at its end and intended to be implanted in the aortic valve position 53 by expansion.

In this embodiment, the suction cup 20 placed around the end of the tube 10 is suitable for being applied to the apex of the heart 2.

Similarly to that which was described above, once the device 1 is positioned on the apex of the heart 2, as visible in FIG. 5a, the vacuum is generated under the suction cup via the suction line 21 so as to fix the device onto the heart, as visible in FIG. 5b.

The tube 10 is then placed in an orientation such that the trocar 51 supporting the heart valve prosthesis 52 is stabilized along the axis compatible with an anatomically and physiologically effective implantation of the heart valve prosthesis in the aortic position 53, as visible in FIGS. 5b and 5c.

Once the tube 10 is placed in a satisfactory position, the vacuum is generated in the sack 30 via the suction line 31, as visible in FIG. 5c. The heart valve prosthesis 52 can then be put in place in the aortic position 53.

In an alternative use of the device according to the invention, heart valve prostheses can be designed and developed for an implantation in the mitral valve position 54 according to the same procedure for intracardiac access with the device according to the invention, or in the tricuspid valve position 55, or in the pulmonary valve position 56.

In a variant that is not illustrated, a device according to the invention is suitable for implanting a hollow needle in an organ so as to inject products therein, for example a medicament or a solution containing modified or cultured cells (engineered cells).

Of course, other variants of implementation, within the scope of those skilled in the art, can be envisioned without departing from the context of the present invention.

1. Device (1) for the implantation of an apparatus (40, 52) on or in a mammalian internal organ, characterized in that it comprises:
a tube (10) for passing the apparatus through,
one end (11) of which is intended to be applied to a site chosen for the implantation of the apparatus, and the other end of which is intended to emerge outside the body of the mammal,
fixing means (20) suitable for fixing the device on the organ and for applying the end of the tube to the chosen site, said means being controlled from outside the body,
rigidifying means (30) suitable for rigidifying the device, said means being controlled from outside the body, so as to fix the position of the tube relative to the fixing means and to the organ, once the device has been fixed on the organ and the end of the tube has been applied to the chosen site by the fixing means.

2. Device according to claim 1, characterized in that the fixing means are a suction cup (20) placed around said end (11) of the tube (10) intended to be applied to the organ, without communicating with the interior of the tube, and provided with a suction line (21) for generating a vacuum under the suction cup so as to be able to cause the suction cup to adhere to the organ and thus to be able to fix the end of the tube at the chosen site on the organ for the implantation of the apparatus.

3. Device according to claim 1, characterized in that the rigidifying means are a circular sack (30) placed around a portion of the tube in the region of the end of the tube intended to be applied to the organ, the sack being closed at its first end (32) around the tube and, at its other end (33), closed over an annular portion (22) of the fixing means (20), the sack being provided with a suction line (31) for generating a vacuum inside the sack, and being designed so that it is flexible when the pressure inside it is the same as the surrounding pressure, and so that, when the vacuum is generated inside it, it tightens around the tube so as to fix the position of the tube that passes through it, relative to the fixing means, and to contribute to rigidifying the device.

4. Device according to claim 3, characterized in that the sack (30) is filled with a plurality of solids (34), free in the sack, so that, when the vacuum is generated in the sack, the sack with the solids tightens around the tube (10) so as to contribute to fixing the position of the tube that passes through the sack, relative to the fixing means (20), and to contribute to the rigidification of the device.

5. Device according to claim 4, characterized in that the sack (30) has bumps on its inner surface, suitable for contributing to fixing the position of the tube (10) relative to the fixing means (20) and to the organ when the vacuum is generated in the sack.

6. Device according to claim 5, characterized in that the sack has bumps on its outer surface, suitable for contributing to fixing the position of the tube (10) relative to the fixing means (20) and to the organ when the vacuum is generated in the sack (30).

7. Device according to claim 7, characterized in that the end of the tube (11) intended to be applied to the internal organ is provided with a ring (12) made of flexible material aimed at making this end non-traumatic.

8. Device according to claim 1, characterized in that the device (1) is intended for a human organ.

9. Device according to claim 1, characterized in that the device is intended for a heart.

10. Device according to claim 9, characterized in that the end (11) of the tube (10) for passing the probe through, and the suction cup (20) placed around the end of the tube, are intended to be applied to the epicardium (3)

11. Device according to claim 10, characterized in that the apparatus to be implanted is a heart valve prosthesis (52).

12. Device according to claim 11, characterized in that:
the apparatus is a trocar guide (51) carrying a heart valve prosthesis (52) set on this trocar guide and intended to be implanted in the aortic valve position (53) by expansion,
the fixing means are a suction cup (20), placed around the end of the tube (10), and suitable for being applied to the apex (2) of the heart,
the position of the tube (10) is intended to be fixed, relative to the fixing means (20) and to the organ, in an orientation such that the trocar supporting the heart valve prosthesis is stabilized along an axis compatible with an anatomically and physiologically effective implantation of the heart valve prosthesis.

13. Device according to claim 12, characterized in that the apparatus to be implanted is a hollow needle designed for injecting a product into the organ.

14. Device according to claim 13, characterized in that the apparatus to be implanted is a cardiac stimulation probe (40).

15. Device according to claim 1, for its use in cardiac therapy.
